(19) United States
(12) Patent Application Publication
(10) Pub. No.: US 2023/0142816 A1
(43) Pub. Date: May 11, 2023

(54) MICROFLUIDIC POINT-OF-CARE ASSAY

(71) Applicant: Mint Diagnostics Ltd, Sittingbourne (GB)

(72) Inventors: Nikolaos VASILAKIS, Sittingbourne (GB); Richard WHITE, Sittingbourne (GB); Stefano BORINI, Sittingbourne (GB); Cedric HURTH, Sittingbourne (GB)

(21) Appl. No.: 17/912,813

(22) PCT Filed: Mar. 12, 2021
(86) PCT No.: PCT/EP2021/056345

§ 371 Date: Sep. 19, 2022

(30) Foreign Application Priority Data
Mar. 19, 2020 (GB) 2003979.8

(51) Int. Cl. B01L 3/00

(52) U.S. Cl. CPC B01L 3/50273; B01L 3/502723; B01L 3/502738; B01L 2200/0673; B01L 2400/0406; B01L 2300/0825; B01L 2300/0645; B01L 2300/161; B01L 2300/087; B01L 2200/0621; B01L 2400/0688

(57) ABSTRACT

The disclosure describes an integrated fluid sample test strip comprising: an inlet for receiving solutions comprising a fluid sample and a substrate solution, the inlet comprising a retention valve for temporarily retaining each solution to thereby reduce air flow through the valve; a reaction chamber to receive the solutions via the retention valve, the chamber functionalized with bioreceptor(s); a capillary pump to receive from the reaction chamber the solution(s), the pump comprising vent hole(s); a test chamber to receive the substrate solution from the reaction chamber, the test chamber comprising test electrodes for a biosensing test of the substrate solution; a hydrophobic vent hole coupled to the test chamber to allow a flow of solution from the reaction chamber into the test chamber when the vent hole is unsealed and to allow a flow of solution from the reaction chamber to the capillary pump when the vent hole is sealed.

### FIELD OF THE INVENTION

The present invention generally relates to an integrated fluid sample test strip, a fluid sample test system, and use of the test strip or test system to perform an ELISA or ELONA test. Preferably the fluid sample comprises saliva. In particular, preferred embodiments measure analyte levels in saliva, for example for hormone testing or monitoring.

### BACKGROUND OF THE INVENTION

Hormones regulate many processes within the body including metabolism, digestion, reproduction, our ability to access energy reserves, our mood and emotions, sleep to name a few. Understanding our endocrinology can help us optimise our health, wellness and/or fitness. For example, cortisol is a stress hormone that is generally released in the human body as a result of physical or psychological stressors. Where the stress response is dysregulated, this can lead to broad health problems and/or performance deterioration, with regard to a person's mental or physical state.

Using for example saliva to test for hormones may be desirable. However, analyte concentrations in saliva can be extremely low. For example, the hormone oestradiol may be present at below picogram/mL levels. Detecting such low concentrations of analytes often requires an assay approach involving amplification. Enzyme-linked immunosorbent assays (ELISA) employ enzymatic amplification to oxidise a substrate species. In the most common form of ELISA the oxidized substrate species are coloured and the intensity of the colour is indicative of the analyte concentration. This approach is known as a colorimetric assay. It is also possible to measure the oxidized substrate species electrochemically to allow greater quantification and sensitivity.

However, to obtain reliable quantitative results from analysis of saliva using existing methods, a sample is generally prepared and controlled prior to analysis. For example, to carry out an ELISA test for detecting cortisol concentration of saliva, the sample is centrifuged and the pH checked before analysis. Similarly, for a salivary lateral flow test, the saliva is diluted in buffer solutions before analysis.

Such existing methods are unsuitable for home saliva testing. Ideally, biosensor devices should be suitable for use by an untrained consumer in their own home. Any requirement for multiple reagents and/or sample pre-treatment generally add complexity to the testing protocol and may introduce significant sources of error, and thus are example barriers to home diagnostic tests becoming more prevalent.

A further barrier is that, to obtain reliable quantitative results from analysis of saliva using existing methods, relatively large volumes of saliva need to be collected. Saliva collection methods such as passive drooling are required with long collection times, e.g., 15 minutes.

The field of fluid sample testing therefore needs an improved method or device to monitor the levels of chosen biomarker(s), e.g., the field of saliva testing needs an improved method or device to monitor such levels in an individual's saliva. Such an improved method may allow, e.g., a compact and/or portable test apparatus, quantitative measurement, greater convenience for the user, speed, accuracy, sensitivity and/or reliability, and preferably without any requirement of a lab environment, trained professionals and/or sample pre-treatment. It is desirable that the testing needs minimum user intervention or input at any stage after the sample collection and during analysis.

For use in understanding the present invention, the following disclosures are referred to:
WO2017/132565 A1: “Saliva Glucose Measurement Devices and Methods” (Labelle et al; published 3.8.17);
US2017/0226557 A1: “Strip-based Electrochemical sensors for Quantitative Analysis of Analytes” (Wang et al; published 10.8.17); and
US2009/0306543 A1: “Specimen Sample Collection Device and Test System” (Slowey et al, published 10.12.09);
U.S. Pat. No. 6,248,598 B1: “Immunoassay that provides for both Collection of Saliva and Assay of Saliva for one or more Analytes with Visual Readout” (Bogema; published 19.6.01)
U.S. Pat. No. 9,223,855 B1: “Method and System for Training Athletes based on Athletic Signatures and a Classification thereof” (Wagner; published 29.12.15);
US20100206748 A1: “Stress Measurement Kit and Stress Measurement Method” (Morita et al; published 19.8.10);
WO2011030093 A1: “Glucose Measurement Method and System” (McColl et al; published 17.3.11);
US2007015286 A1: “Diagnostic Strip Coding System and Related Methods of use” (Neel et al; published 18.1.07);
US2011174616 A1: “Methods for Measuring Physiological Fluids” (Roberts et al; 21.7.11);
US2016313313 A1: “Lateral Flow Assay Apparatus and Method, and Sensor Therefor” (Love et al; published 27.10.16);
JPWO2014181753 A1: “Measuring Device” (Shigeru; published 13.11.14);
US2011108440 A1: “Underfill Recognition System for a Biosensor” (Wu et al; published 12.5.11);
“The lab-on-PCB approach: tackling the μTAS commercial upscaling bottleneck” (Moschou et al; Lab Chip, 2017, 17, 1388. DOI: 10.1039/c71c00121e);
“ELISA-type assays of trace biomarkers using microfluidic methods” (Dong et al; WIREs Nanomed Nanobiotechno. 2017, 9:e1457. DOI: 10.1002/wnan.1457);
“Materials for Microfluidic Immunoassays: A Review” (Mou et al; Adv. Healthcare Mater. 2017, 6, 1601403. DOI: 10.1002/adhm.201601403);
“A Novel Microfluidic Point-of-Care Biosensor System on Printed Circuit Board for Cytokine Detection” (Evans et al; Sensors 2018, 18, 4011; DOI:10.3390/s18114011);
WO2017025921A1: “Aptamer Biosensors useful for detecting hormones, hormone mimics, and metabolites thereof” (Kumar, published 16.2.17);
U.S. Pat. No. 9,207,244B2: ‘System and methods for detection and quantification of analytes’ (Khatak et al; published 21.5.15);
Zimmerman et al, LabChip, 2007, 7, 119-125, “Capillary pumps for autonomous capillary systems”.
G. Volpe et al, Analyst, June 1998, Vol. 123 (1303-1307).

### SUMMARY

According to the present invention, there is provided an integrated fluid sample test strip comprising:
i. an inlet for receiving a series of solutions, said solutions comprising at least a fluid sample and a substrate solution, wherein the inlet comprises a retention valve for temporarily retaining each said solution to thereby reduce air flow through the retention valve;
ii. a reaction chamber to receive the solutions from the inlet via the retention valve, the reaction chamber functionalized with one or more bioreceptors for binding to a target analyte;
iii. a capillary pump for receiving from the reaction chamber at least one of the solutions including at least the fluid sample, the capillary pump comprising at least one vent hole to allow any air to escape from the capillary pump and thereby reduce pressure in the capillary pump;
iv. a test chamber to receive the substrate solution from the reaction chamber, the test chamber comprising a plurality of test electrodes to perform at least part of a biosensing test of the substrate solution;
v. a hydrophobic vent hole coupled to the test chamber to allow a flow of solution from the reaction chamber into the test chamber when the vent hole is unsealed and to allow a flow of solution from the reaction chamber to the capillary pump when the vent hole is sealed.

Preferred embodiments may provide advantages such as accuracy, sensitivity and/or reliability of detection or measurement of a target analyte, e.g., biomarker, that may be in received saliva. This is desirable in various fields of activity, such as health or wellness programs for elite sports training and/or precision medicine to allow drugs administration tailored according to a patient, e.g., to their individual hormone levels. Preferably, the integrated nature of the strip allows all of the features i-v to be provided to the user within a one-piece device that is preferably portable, compact and/or disposable.

While the present disclosure generally refers throughout to assays for testing saliva, e.g., salivary hormone tests, embodiments may additionally or alternatively be suitable for use with one or more other matrices. Such other matrices which may comprise bodily fluids such as blood and/or urine. Further example matrices comprise non-bodily fluids, e.g., water, for example for use in environmental monitoring.

Therefore, references to saliva within the present disclosure are generally interchangeable with references to any one or more of such other fluids.

The test strip may enable incubation of a sample and a competing conjugate with bioreceptor molecules (such as an antibody or aptamer) in the reaction chamber, followed by a subsequent electrochemical measurement in the test chamber. The incubation of the sample and competing conjugate may be simultaneous or sequential. The architecture may enable precise control of liquid within the microfluidic network, allowing the measurement of analyte levels in an, e.g., saliva, sample in a relatively small amount of time (for example, in less than 1 hour). Embodiments may not require a laboratory setting for operation of the strip and/or for preparation of the saliva sample, and therefore may advantageously allow analysis in a field or point-of-care environment.

The inlet may allow solutions and other fluids to be introduced to the test strip (noting that the term ‘fluid’ is used herein to refer to ‘liquid’, e.g. a solution). Preferably (i.e., optionally), the inlet is configured to have an external aperture cross-section that is sufficiently wide to ease the introduction, yet is of small enough width (e.g., diameter) to allow dispensed fluids to wet preferably an entire bottom surface of the inlet. Advantageously, a large inlet width/diameter may minimise capillary effects near the external aperture of the inlet, while allowing preferably the entire bottom of the inlet to be wetted by introduced fluids. This may ensure that preferably all of the fluids will enter the microfluidic network of the test strip. The width (e.g., diameter) and/or volume of the inlet may therefore vary between different embodiments, depending on the volume of fluids required fora particular assay. The inlet volume may be determined such that an expected combined volume of introduced fluids is greater than the combined internal volume for holding fluid in the test strip downstream of the retention valve. (Added volume preferably exceeds a total volume of the test strip including the test chamber. If added volume is less than that total volume then the test chamber may in embodiments pull fluid from capillary pump end potentially causing errors. In practice however the inlet generally does not empty after filling the test chamber). This may allow one or more later introduced fluids to be retained in the inlet. Advantageously, such retention of fluid in the inlet may provide additional hydrostatic pressure, which may thereby assist flow of solution from the reaction chamber into the test chamber once the seal of the hydrophobic vent hole is opened.

The retention valve may reduce (e.g., prevent) air flow into downstream components of the strip, e.g., into the reaction chamber and/or test chamber. This may improve the accuracy, sensitivity and/or reliability of testing. The retention valve may be active or passive, wherein a passive retention valve may have the greatest capillary pressure in substantially (preferably entirely) the whole capillary system of the test strip, e.g., not just higher relative to (immediately) adjacent microfluidic features. In embodiments, the retention valve generally pins the dewetting meniscus. An example passive valve generally comprises a fluid flow path having a constriction, and/or a narrow path relative to at least directly adjacent features from which the fluid flows in and out of the valve, and/or preferably has a smaller cross sectional area for fluid through-flow than any other microfluidic features of the test strip. The valve may be configured to regulate flow of liquid through the valve by means of capillary force.

More specifically, the retention valve may ensure fluidic flow with reduced (preferably complete absence of) air bubbles downstream in the test strip (e.g., in any fluid in flow paths between the retention valve and the capillary pump or test chamber), and/or may reduce or prevent ‘dead’ volumes where non-specific reactions or binding events (which may be other than those between the analyte of interest and the bioreceptor molecules (antibody and/or aptamer)) may otherwise take place in the test strip. Non-specific reactions or binding events are generally undesirable and may be due to species sticking to the channel surfaces or solution getting trapped in a non-preferred location. Such dead volumes may increase the noise in measurement values, decreasing the accuracy and/or reliability of the test results. Preferably, the inlet retention valve is configured to have the greatest capillary pressure in the test strip. This may be enabled, for example, by the retention valve having a smaller cross-sectional area than any of the other components of the test strip. This may result in the valve rarely (preferably never) becoming empty of fluid during use of the test strip. (Noting that the term ‘cross-section’ is used herein to refer to an area through which can flow).

In embodiments, at least one biosensing test may comprise at least two stages: biorecognition; and an electrochemical transduction. The biorecognition may occur in the reaction chamber, while the electrochemical transduction may occur in the test chamber (alternatively referred to herein as a measurement chamber or sensing chamber). The reaction chamber may be pre-functionalised with bioreceptor molecules during manufacture of the test strip. Such bioreceptor molecules may bind with a target analyte in fluids such as solutions introduced into the test strip. This binding may occur due to the biorecognition between the bioreceptor molecule and the target analyte. This may allow test strips to be designed to detect levels of specific target analytes, reduce the number of steps required by the user, and/or improve the reliability of the results in point of care use.

The test chamber may be configured to expose solutions to one or more test electrodes. Such test electrode(s) may be controllable to perform the electrochemical transduction and detect a level of the target analyte in a sample input into the test strip. In this regard, the test strip may be used for sequential and/or simultaneous competition immunoassays. In such embodiments, the electrochemical transductions may be performed on one or more later input fluids (e.g, solutions), rather than directly on a test (e.g., saliva) sample. Additionally or alternatively to sequential and/or simultaneous competition immunoassays, the test strip may be used for other assay formats. For steroid hormones, which are small molecules, a competitive assay may be preferred, for example because each hormone molecule generally only has one epitope.

The test electrodes may enable an electrochemical test of a substrate solution. This may facilitate extremely sensitive quantification of, e.g., salivary, hormone concentrations. In embodiments, the hydrophobic vent hole may be sealable and/or unsealable to control flow of fluid(s) into the test chamber (noting merely for completeness that if the pump is full then flow to the pump may be prevented regardless of the seal). This may allow reduction (e.g. prevention) of flow of unwanted fluids into the test chamber. In embodiments, the hydrophobic nature of the vent hole may reduce (preferably stop) flow of fluid into or within the test chamber, for example when the test chamber is already full. A reduction in the flow rate of solution in the test chamber may improve accuracy of an electrochemical test of a solution, for example by ensuring that the measurement regime is at least approximately diffusion controlled, or is dominated by diffusion. In a preferred implementation, the hydrophobic vent hole (and/or any vent channel connected thereto, e.g., to connect a test chamber to the hydrophobic vent hole) has a PTFE (Polytetrafluoroethylene) coating to provide the hydrophobicity.

The capillary pump may allow waste solution(s) to flow through the test strip and away from the microfluidic elements involved in performing the biosensing test, e.g., the reaction chamber and/or test chamber. The capillary pump may comprise one or more vent holes to allow air in the test strip to evacuate the capillary pump as input fluids flow through the strip. Advantageously, such vent hole(s) may reduce, e.g., prevent, any increase in air pressure in the test strip. An increase in air pressure (due to, for example, trapped air) may reduce the flow rate of the input fluids and/or prevent the fluids from flowing through the test strip.

There may further be provided a branched flow path to guide solution from the inlet to the hydrophobic vent hole and from the inlet to the capillary pump, wherein the capillary pump comprises at least one capillary channel, the branched flow path comprising at least the elements i-v including the at least one capillary channel, wherein a smallest cross-sectional area of the branched flow path is a cross-sectional area of the retention valve. Such solution may comprise substrate solution to flow from the inlet to the hydrophobic vent hole and/or fluid sample such as saliva to flow from the inlet to the capillary pump. The smallest cross-sectional area may be an area through which all such solutions must flow to reach the reaction chamber, and may be relative to all other cross-sectional areas for fluid flow through the branched flow path. The branched flow path may advantageously allow improved regulation of flow of fluids through the test strip and/or reduce any risk of contamination of the test chamber with unwanted fluids. For example, the branched path configuration in conjunction with the hydrophobic vent hole may allow a higher degree of control of which solution (preferably only the substrate solution) flows into the test chamber and/or control of which solutions flow into the capillary pump.

There may further be provided a test strip wherein the reaction chamber is configured to incubate a solution and the test strip comprises a further retention valve for temporarily retaining a said incubated solution. Such a further retention valve may provide one or more of the advantages of the inlet retention valve, include, e.g., allowing fluidic flow without the formation of air bubbles in test strip and/or reducing ‘dead’ volumes. Preferably, the further retention valve may be configured to have a capillary pressure exactly or approximately equal to a capillary pressure of the inlet retention valve.

There may further be provided a test strip wherein the capillary pump comprises at least one capillary channel defined by an array of micropillars. Such a channel may provide capillary pressure, which may enhance or enable flow of solutions into and/or through the capillary pump.

In some embodiments, at least one said micropillar may comprise a substantially diamond-shaped cross section. Such a shape may be exactly diamond shaped, or may for example have curved corners. A diamond shape may be substantially (e.g., exactly) rhombic.

There may further be provided a test strip wherein the capillary pump comprises an internal bypass channel along at least part of a perimeter of the capillary pump, wherein a smallest cross-sectional width of the bypass channel is greater than a smallest separation between adjacent said micropillars. Bypassing flows may be reduced in such an embodiment. Such a bypass channel in the form of a peripheral clearance between a boundary of the capillary pump and the array of micropillars may reduce or prevent bypassing flows along the frame of the micropillar array.

There may further be provided a test strip wherein a smallest separation between adjacent said micropillars is less than a smallest width of a solution flow path from the reaction chamber to the capillary pump. In embodiments this may provide a greater capillary pressure in the capillary pump compared to the flow path between the reaction chamber and capillary pump. Advantageously, this may provide a more robust flow of fluids into the capillary pump.

There may further be provided the test strip, wherein the capillary pump has an inlet comprising a constriction. The introduction of a bypass channel may result in a gap between the micropillar array and the pump inlet, and this gap may reduce or prevent flow into the pump. The constriction may reduce the gap between the capillary pump inlet and the micropillar array, and may thereby maintain a capillary pressure at the inlet of the capillary pump and thus aids flow into the pump.

There may further be provided a vent hole channel, wherein the hydrophobic vent hole is coupled to the test chamber via the vent hole channel to allow any air in the test chamber to escape to thereby reduce pressure in the test chamber. Such a hydrophobic vent hole may increase time required for the test chamber to fill with fluid. A vent hole channel may however reduce the direct effect of the hydrophobic vent hole on fluids in the test chamber itself, preferably minimising the effect of the hydrophobic vent hole on test chamber fill times.

There may further be provided a test strip comprising at least one of: a hydrophilic layer, wherein at least one surface of the hydrophilic layer is hydrophilic; and a polymer layer.

There may further be provided a test strip wherein:
the test chamber is formed in at least the hydrophilic layer;
a channel for guiding solution from the reaction chamber to the capillary pump is formed in at least the polymer layer;
the inlet is formed in at least the polymer layer;
the capillary pump is formed in at least the polymer layer; and/or
at least one said vent hole is formed in at least the polymer layer.

Thus, features of the test strip may in some embodiments be formed within the above-mentioned hydrophilic and/or polymer layers.

There may further be provided a passive stop valve to at least reduce a flow rate of solution into the test chamber. Such a stop valve may reduce unwanted flow into the test chamber, and this may reduce the risk of contamination of the test chamber and/or increase the reliability and/or accuracy of test results.

The test strip may be configured to measure levels of the analyte, wherein the analyte is a hormone. More generally, the analyte may comprise, e.g., be, a hormone and/or biomarker. The test strip may be configured to perform the measurement by performing an ELISA or ELONA (Enzyme-Linked Oligonucleotide Assay) test, and/or other tests.

There may further be provided the test strip, wherein the fluid sample comprises saliva, blood or urine. According to another aspect of the invention, there is provided a fluid sample test system comprising the fluid sample test strip and at least one of:
a fluid sample collector device for collecting the fluid sample and inputting the fluid sample into the inlet; and
a reader device for controlling at least one of the test electrodes to perform the at least part of the biosensing test, and to output a result of the biosensing test.

Such a reader device may be used to determine and/or output analyte levels from raw test result data. Additionally or alternatively, a fluid sample collector device may be configured to aid in the collection of, and/or input into the inlet of, the fluid sample by the user.

According to a further aspect of the invention, there is provided a use of the fluid sample test strip or the fluid sample test system, to perform an ELISA or ELONA test.

There may further be provided the use, comprising:
(i) receiving the fluid sample in the inlet;
(ii) receiving the substrate solution in the inlet; and/or
(iii) unsealing the vent hole.

There may further be provided the use, comprising:
(iv) receiving a solution comprising an enzyme-conjugate in the inlet; and/or
(v) receiving one or more wash-buffer solutions in the inlet.

### BRIEF DESCRIPTION OF THE DRAWINGS

For a better understanding of the invention and to show how the same may be carried into effect, reference will now be made, by way of example, to the accompanying drawings, in which:

FIG. 1 shows a schematic of a saliva test strip according to an embodiment of the invention;

FIG. 2 shows a schematic of layers of a saliva test strip embodiment of the invention;

FIGS. 3a and b shows an enhanced view of parts of the test strip of FIG. 1;

FIG. 4 shows an exploded view of layers of the saliva test strip according to an embodiment of the invention;

FIG. 5 shows an example cross section of the reaction chamber and an example cross-section of the measurement chamber of embodiments of the invention, wherein either or both of which chambers is preferably a microfluidic chamber;

FIG. 6 shows schematically an example construction of a preferably second layer of the test strip embodiment;

FIG. 7 shows an image of an assembled test strip according to an embodiment of the invention;

FIG. 8 illustrates an example competitive assay comprising steps a-d.

FIG. 9 shows a flow chart of an example method of measuring analyte concentrations in a saliva sample according to an embodiment of the invention;

FIG. 10a-f illustrates an example flow of fluid through an embodiment of the invention during the method of FIG. 9.

FIG. 11 shows a block diagram of an example reader device;

FIG. 12 shows a block diagram of an example collector device; and

FIG. 13 shows a block diagram of an example test system.

### DETAILED DESCRIPTION OF EMBODIMENTS

Embodiments generally provide a microfluidic apparatus for performing an assay, such as an enzyme-linked immunosorbent Assay. The assay is preferably performed at a point of care, which is generally a location that is convenient to the user and thus preferably not in a dedicated a laboratory.

In general, a competitive immunoassay is one where the analytes (e.g. hormone molecules) in a sample (e.g. saliva, blood, urine) and a fixed amount of a labelled analyte analogue (e.g. the “conjugate”; analyte conjugated with a radioisotope, fluorescent or enzyme label) compete for the binding sites on a film with a known amount of immobilised antibody. Once the sample, conjugate and antibodies have been incubated together and the competition has taken place, the amount of analyte is determined by measuring the amount of conjugate that has bound to the antibody (or alternatively, that remains free in solution).

In some embodiments of the invention, a conjugate comprising the hormone of interest and an enzyme such as horseradish peroxidase (HRP) is used. In the presence of a substrate solution (such as hydrogen peroxide with tetramethylbenzidiene (TMB)), HRP may oxidise the TMB. At lower analyte concentrations in the sample, the antibodies bind to a higher proportion of conjugates. As a result, when the substrate is incubated with this antibody film the TMB will oxidise to a greater extent. Oxidised TMB is typically measured colorimetrically (for example, TMB may turn blue or yellow depending on the degree of oxidisation). However, embodiments of the present invention may measure the TMB electrochemically in order to increase the sensitivity of test. (see ref. Analyst, June 1998, Vol. 123 (1303-1307) by G. Volpe et al.). (For further detail regarding TMB, it is noted that a blue product of a HRP/H₂O₂+TMB reaction is generally a one-electron oxidation product of TMB. A two-electron oxidation product is generally coloured yellow. After the HRP reaction with TMB/H2O2, the reaction may be stopped by using a strong acid, which may further oxidise the one-electron oxidation products and/or stabilise the system preferably to allow more accurate measurement in a spectrophotometer or plate reader. However, use of a stop solution in embodiments of the present invention may displace reacted TMB from the reaction chamber. Preferably, embodiments do not use a stop solution and/or measure the one-electron oxidation product).

Generally, there are two approaches that can be employed for a flow-based competitive immunoassay, either sequential or simultaneous competition. In simultaneous addition, the sample is mixed with the conjugate solution and then they are incubated with the antibody film simultaneously. In sequential addition, the sample is introduced to the antibody film first and has the first opportunity to bind with the antibody binding sites and then the conjugate solution is introduced afterwards to react with the remaining binding sites. Embodiments of the present invention are suitable for either approach. However, in some embodiments sequential addition is preferred as this approach generally enables a higher sensitivity and doesn't require the preparation of a sample/conjugate solution with precise volumes mixing prior to the testing procedure.

FIG. 8 shows an example of a sequential addition competitive immunoassay in an embodiment. In step A, a sample (for example, saliva) containing a target analyte (for example, a hormone) binds with some of the bioreceptor molecules (for example, an antibody). In step B, an enzyme conjugate (for example, HRP) binds with some or all of the remaining bioreceptor molecules. In step C, a buffer solution is used to remove the remaining unbound enzyme conjugate from the vicinity of the bioreceptor molecules. In step D, a substrate solution (hydrogen peroxide with tetramethylbenzidiene (TMB)) reacts with the bound enzyme conjugate. In general, the amount of reacted or oxidised substrate solution may be related to the amount of bound enzyme conjugate. As a result, in this example, the greater the measured oxidation of the TMB the lower the hormone analyte concentration in the saliva sample.

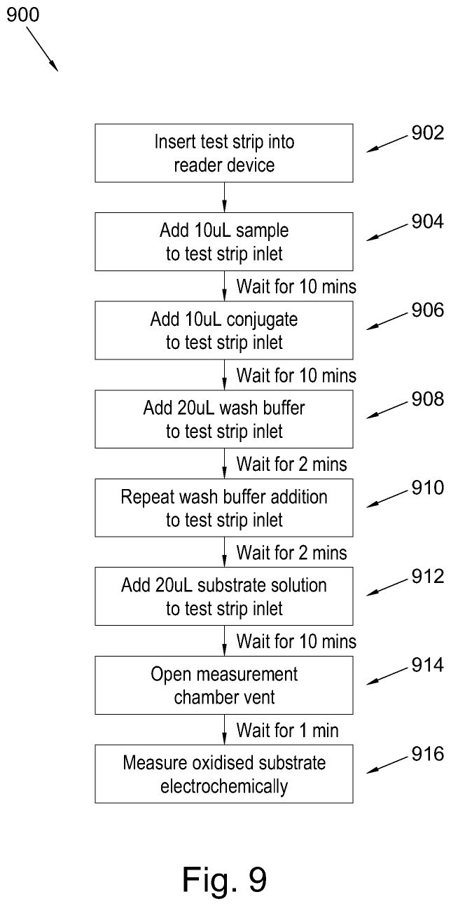

FIG. 9 shows an example process 900 for performing a sequential addition competitive immunoassay, such as an ELISA test, using a test strip according to an embodiment. In step 902 the test strip is inserted into an electronic reader device (however this step could alternatively occur as a final or intermediary step in the process). The reader device may be used to, for example, control the test electrodes and/or provide the test results to the user.

In step 904 a sample containing a target analyte is inserted into the test strip via the inlet. This sample may be, for example, a 10 μL saliva sample from the user. The volume of the sample may vary depending on the particulars of e.g. the immunoassay and target analyte.

In step 906 a conjugate solution is inserted into the test strip via the inlet. In alternative embodiments, for example embodiments of the test strip which utilise a simultaneous competition immunoassay, the conjugate may be mixed with the sample and inserted into the test strip in step 904. The conjugate solution may be, for example, a 10 μL solution containing target analyte conjugated with an enzyme. As with the sample in step 904, the volume may vary depending on the particulars of the test.

In step 908 a wash buffer solution may be inserted into the test strip via the inlet. The wash buffer helps to prevent contamination of the later substrate solution by the conjugate solution and/or by unbound enzyme-conjugates in the reaction chamber. By reducing the probability of contamination the wash buffer solution may improve the reliability of the results. In some embodiments, the wash buffer solution may have a volume of about 20 μL. In optional step 910, additional wash buffer solutions may be used to further reduce the risk of contamination of the substrate solution. The additional wash buffer solutions may have a volume the same as or different from that of the initial wash buffer solution.

In step 912 a substrate solution may be inserted into the test strip via the inlet. The substrate solution and/or volume used may depend on the target analyte and/or enzyme conjugate. In this example process, the substrate solution may have a volume of about 20 μL.

In step 914 the test chamber vent is opened. Opening this vent may allow air to escape from the branched microfluidic system (including the test chamber). This may in turn allow the substrate solution to replace air in the branched system.

In step 916 the user controls the test electrodes in the test chamber to measure the substrate solution. This measurement may be accomplished by using electrochemical transduction to measure the amount of oxidised substrate solution. As discussed above, the test electrodes may be controlled via a reader device, which may then present the results to the user.

Between any two of the above steps the user may allow a certain amount of time for the fluids to propagate through the test strip. Possible wait times between each step are shown in FIG. 9. For example, the wait time between each of steps 904, 906 and 908 may be about 10 minutes, while the wait time between each of steps 908, 910 and 912 may be about 2 minutes. Similarly, the wait time between steps 912, 914 and 916 may be about 10 minutes and about 1 minute respectively. The user may be prompted to proceed to the next step by the reader device, and/or be provided with instructions (separately or by the saliva test system) detailing any such wait time(s).

The volumes and/or incubation times discussed with regard to example process 900 can vary. For example, the short (e.g., 2 minute) wait times may allow each added reagent solution to flow into the test strip channels and/or ensure that the next reagent is added to an empty inlet, thereby reducing or avoiding uncontrolled dilution in the inlet. The longer (e.g., 10 minute) incubation times may be set according to the specific antigen-antibody reaction times. Such times may be between about 5 minutes and about 30 minutes. In some embodiments, reactions in the microfluidic channels may reach equilibrium conditions in a shorter period of time than reactions in e.g. in standard plate wells, due to reduced channel dimensions and subsequently shorter diffusion lengths.

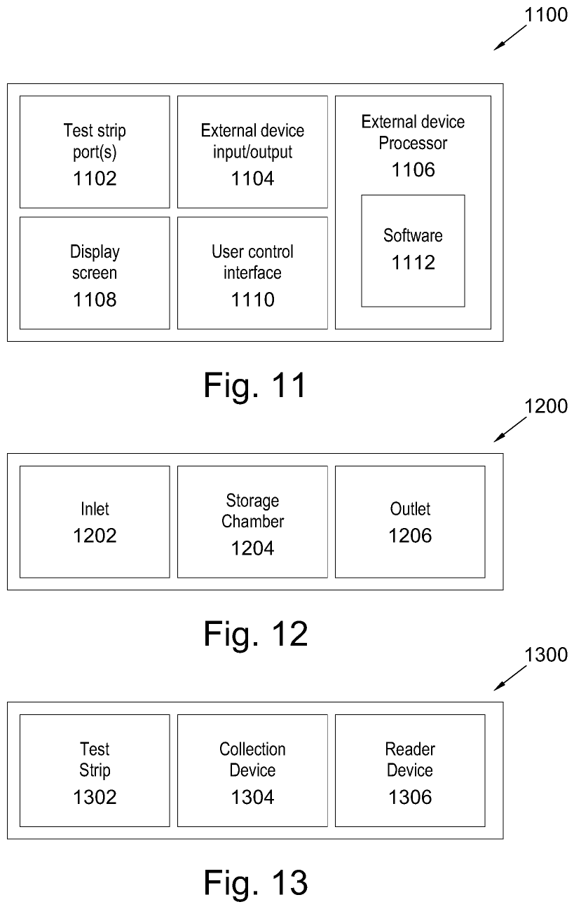

FIG. 11 shows a block diagram of an example reader device 1100. The reader device may be any device capable of controlling the test electrodes to perform an electrochemical measurement of the substrate solution, processing of raw test data into analyte concentration and the communication of the analyte concentration to the user, for example via a host computer or mobile phone, and/or by a display on the reader device. Optionally, the reader device may control the test chamber vent to open, for example by actuation. This actuation may be automatic, or it may be controlled by the user through a user control interface 1110, for example a button or a touch screen display. In one embodiment the reader device is a preferably USB-connected multi-channel potentiostat device that may be controlled by software located on a computing device. In this embodiment, the reader device may have a component 1104 configured to communicate with external computing devices, such as a USB port. The reader device may only have one test strip port 1102, however in some embodiments the device may have multiple ports 1102 to enable multiple test strips to be operated simultaneously. For example, a device with 5 ports would enable simultaneous testing with 5 test strips. The reader may be provided with a processor 1106 running software 1112 to guide the user through the multi-step protocol, and/or this functionality may be provided by software on the computing device. The reader device may further be provided with a display screen 1108 for providing information to the user, and/or this functionality may be provided by an external computing device.

Such a process may be implemented using test strip embodiments as described in the following. Such embodiments may allow multiple reagent solutions to be added to a test strip in a simple way and/or without the need for any active pumping mechanism. In this regard, one way to achieve passive pumping of fluids is to utilize capillary pressure. Zimmerman et al (LabChip, 2007, 7, 119-125, Capillary pumps for autonomous capillary systems) defines the capillary pressure Pc of a liquid-air meniscus in a microchannel as:

$${P}_{c} = - γ ( \frac{cos {α}_{b} + cos {α}_{t}}{a} + \frac{cos {α}_{1} + cos {α}_{r}}{b} )$$

where γ is the surface tension of the liquid, a_{b,t,l,r} are the contact angles of the liquid on the bottom, top, left, and right wall, respectively, and a and b are the depth and width of the microchannel, respectively. Microfluidic components typically have sub-millimeter dimensions and thus may allow for precise control and manipulation of fluids via capillary action. References to microfluidic channels and/or chambers throughout the description generally refer to channels and/or chambers of dimensions at which the mass transport of fluids is primarily governed by capillary pressure. References to capillary pressure herein generally relate to capillary pressure of a saliva-air interface, and may be approximated based on the above equation for capillary pressure Pc based on an assumption that the relevant structure, e.g., channel or chamber, can be approximated as having a substantially rectangular cross-section.

The above definition of a liquid-air capillary pressure Pc may generally be applied to references to capillary pressure throughout the present disclosure, for example in relation to the inlet retention valve having a greatest capillary pressure in the test strip, which may in turn specifically relate to capillary pressure of a saliva-air interface. In embodiments, liquid in the channel may be pulled from each end by the capillary pressure (or combined surface tension) at the liquid-air interface. Liquid may flow into the capillary pump when there is liquid in the inlet since the pull from the inlet may be less than the pull from the capillary pump. (In addition to this capillary pressure difference, liquid in the inlet with height greater than the depth of the channels may generally exert a hydrostatic pressure). Once the inlet is empty and the upstream liquid-air interface is located in the retention valve then the pull there is generally greater than the capillary pump pull so it becomes pinned there. Inlet and test chamber retention valves jointly may have the greatest capillary pressure in a test strip embodiment.

FIG. 1 shows an example saliva test strip 100, that having an inlet that includes an inlet port 102 and a microfluidic retention valve 104 immediately adjacent to inlet port 102. The inlet retention valve 104 may at least temporarily maintain a position of fluid within retention valve 104 when inlet 102 is at least substantially (e.g., fully) otherwise empty of input solution(s). Advantageously, the retention valve may pin the fluid in position once the inlet is effectively empty. The retention valve 104 may help to reduce or avoid bubble formation in a microfluidic channel leading toward the reaction chamber 106, even upon multiple serial additions of solutions. Inlet 102 is connected to the microfluidic reaction chamber 106 via inlet retention valve 104 and the microfluidic channel. The reaction chamber 106 may be used for incubation or reaction of solution(s) such as saliva, with bioreceptor molecules. Reaction chamber 106 (alternatively referred to as an incubation chamber) may be pre-functionalised by dispensing bioreceptor molecules along one or more of its surfaces. The bioreceptor molecules may comprise, for example, antibodies to bind with a target hormone analyte in a saliva sample. Test strip 100 further comprises a capillary pump 110 that is connected to reaction chamber 106 by a microfluidic channel and may comprise vent hole(s) 112. Preferably, the capillary pump 110 has a capillary pressure greater than the rest of microfluidic circuit of test strip 100 except for any microfluidic retention valve(s).

The test strip 100 may further comprise a side microfluidic circuit that branches off from the main fluidic channel between reaction chamber 106 and capillary pump 110. Such a side microfluidic channel may include a passive microfluidic stop valve 114. Stop valve 114 may reduce, e.g., prevent the formation of air bubbles within the test strip by ensuring that fluid in the side microfluidic channel remains in contact with fluid in the main branch and preferably preventing bubble formation here. In some embodiments, stop valve 114 may also reduce unwanted flow of solution from the main fluidic channel. The side microfluidic channel may include a second microfluidic chamber referred to as test chamber 116. This test chamber 116 may be used for determining hormone and/or other analyte levels in a sample by performing a measuring or sensing test on a solution such as a substrate solution. For example, as discussed in more detail with reference to FIG. 8 above, a measurement of a level of oxidization of a substrate solution (e.g., a measure of an amount of oxidised TMB, which may be indicative of concentration of hormone or other analytes) may be used to determine a corresponding analyte level in a test sample. (Embodiments may not measure hormones directly). Test chamber 116 (alternatively called a measurement and/or sensing chamber) may allow electrochemical measurement of such a solution by exposing the solution to test electrode(s). Test chamber 116 may be formed as a cavity in an adhesive film laminate such that test electrode(s) are exposed only in this region. Test chamber 116 may further include a vent channel 120 that connects the test chamber 116 to a hydrophobic vent hole 118. The hydrophobicity of the vent hole 118 may act to temporarily stop (i.e., pause) or slow down the flow of solution after the solution has been transferred from the reaction chamber 106 into the test chamber 116; this may improve the accuracy of any measurements. Preferably, the duration of the pause is sufficient to allow measurements to take place under no-flow conditions. Vent hole 118 preferably pauses the flow at a position where a known volume of fluid has passed from the reaction chamber 106 into test chamber 116. In the absence of hydrophobic vent hole 116, the solution may continue to flow by wetting the walls of vent hole 116 and eventually may also reach the outer surface. Such a continuous flow condition may be detrimental to the measurement because the solution may keep flowing over the electrodes during the measurement, as the measurement regime is no longer diffusion limited. Additionally or alternatively, the continuous flow may cause some solution which was not incubated in reaction chamber 106 to be measured, further affecting the accuracy of the results.

The side microfluidic circuit may be positioned to minimise the distance between a reacted or incubated portion of a substrate solution in the reaction chamber 106 and the inlet to the test chamber 116. Similarly, the branching circuit may be positioned at a distance from the reaction chamber that is sufficient to reduce any disturbances of the flow within the reaction chamber caused by the side circuit. This may ensure that the functionalisation chemistry (such as the bioreceptor molecules) applied to the reaction chamber preferably during manufacture of the strip do not enter or make contact with the inlet to the test chamber.

Hydrophobic vent hole 118 may initially be sealed to prevent or reduce flow into the test chamber 116. The hole 118 may opened by, for example, piercing or removing a film to initiate flow after the substrate has incubated with the bioreceptors in the reaction chamber 106.

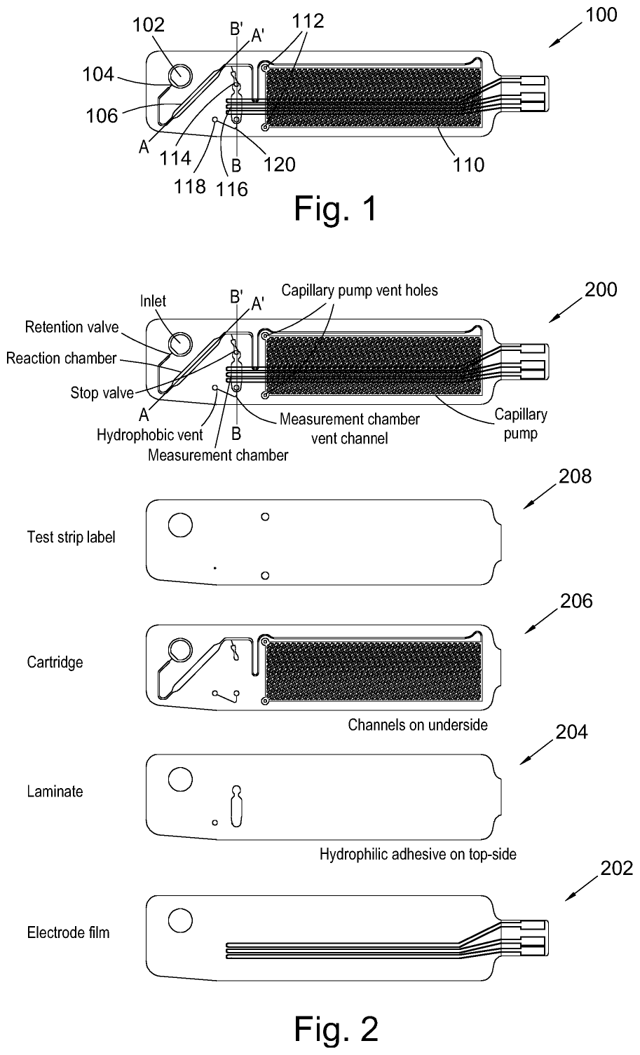

FIG. 2 shows an example structure of an embodiment such as test strip 100 or 200. The test strip comprises a stack formed of a plurality of layers. Example such layers are described below as first to fourth layers, however any one or more of those layers may not be present, and/or additional layer(s) may be provided above, below or in-between those layers.

First layer 202 may be an electrode film, for example a Au/PET film, for example a preferably sputtered gold film with a thickness of, e.g., about (i.e., exactly or approximately) 20-1000 nm on a PET film. In one embodiment a thickness of about 50 nm is used, and/or the Au/PET film has a square resistance of 5Ω/□. The gold may be patterned for example by laser ablation to form an electrical circuit for use in an electrochemical measurement. Alternatively or additionally, the electrodes may be screen printed electrodes or part of a circuit formed by lithographic processes.

Second layer 204 may be a laminate layer such as a 2-ply laminate stack of a double-sided adhesive film (such as PET with inert, acrylic, pressure-sensitive and/or medical-grade adhesive on each side) with a preferably single-side adhesive film (such as PET with preferably hydrophilic and/or pressure-sensitive adhesive). In another embodiment, laminate layer 204 may comprise a single preferably double-sided adhesive film, where the adhesive on at least one side may be hydrophilic. The total thickness of laminate stack 204 may define the volume of the test chamber. The laminate stack may have a thickness of between about 5 μm and about 300 μm. More specifically, an example thickness may be approximately 200 μm, e.g., 183 μm.

Third layer 206 may comprise a microfluidic cartridge such as a PMMA layer. The PMMA layer may have a thickness of greater than about 0.5 mm. For example, a preferred embodiment may have a thickness of at least 2 mm. The microfluidic channels may be formed by laser ablation, injection moulding and/or hot embossing. Additional or alternative polymers may include cyclic olefin polymer (COP), cyclic olefin copolymer (COC), polycarbonate (PC) and/or polystyrene (PS). The depths of the channels in this layer may be less than or about 300 μm. However, a total thickness of at least 2 mm may be preferred to provide a sufficient inlet volume such that dispensed reagent volumes may be confined. Alternatively, a thinner PMMA cartridge may have a wider inlet area to compensate for the lost volume. The depths of the channels along with the channel width and length may define the volume capacity of the test strip, and the reagent volumes may be set accordingly. (Thickness of the cartridge may be driven by volumes such as capacity of the test strip in total, balance between reaction and test chamber volumes, and/or maximum reagent volume to be added to the test strip. In embodiments, the channels themselves may be less than about 0.3 mm deep so that the strip could then be about 0.5 mm thick to robustly accommodate these. However, for a preferred volume, e.g., up to 40 uL, of reagent volume may be added to the inlet, the inlet capacity is preferably suitable to hold this without overflowing. A thinner PMMA strip of thickness, say, about 0.5 mm and a separate inlet apparatus may increase the capacity there).

Fourth layer 208 may comprise a test strip label and may be a printed and/or die-cut PVC (vinyl) or PET (polyester terephthalate) label. The test strip label may include test strip information and/or branding for the test strip, and/or may form a seal over the test chamber vent hole. Such a seal may be broken in order to open the vent hole, for example by piercing, peeling off or otherwise removing the label film. The label 208 may screen the reaction chamber from exposure to light. This may be advantageous, as light exposure can oxidise a TMB substrate and reduce accuracy of any measurement.

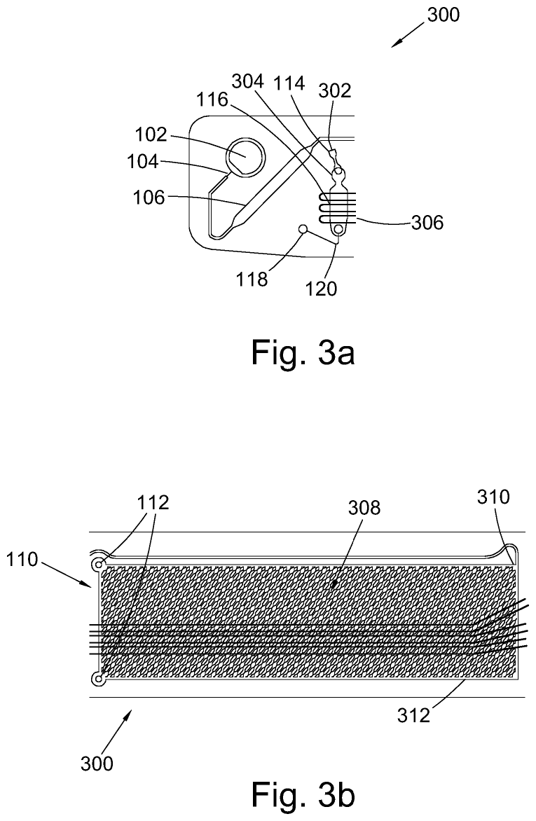

FIGS. 3a and 3b show an enhanced view of a part 300 of a test strip such as test strip 100 or 200. Feature correspondence with features of FIG. 1 is shown by corresponding numerals. The diameter of inlet 102 may be selected based on the assay reagents volumes. In general, the inlet is preferably as wide as possible to minimise capillary effects. However, the inlet diameter preferably further ensures that the dispensed volumes, i.e., input fluids such as the saliva and other solution(s), will generally (preferably always) wet the entire bottom of the inlet. This may reduce the risk that input liquid does not enter the microfluidic network. For example, the inlet may have a width, e.g., diameter of, about 4 mm, e.g., 3.9 mm. This width may be higher or lower depending on factor(s) such as a desired volume to receive inserted solutions and/or the thickness of the test strip.

The microfluidic retention valve 104 is connected (preferably directly) to an internal aperture of inlet 102. Retention valve 104 may also be connected directly or indirectly to a microfluidic inlet channel to guide fluids to reaction chamber 106. This valve may comprise a microchannel and/or may have a width of between about 1 μm and about 500 μm and a length of about 0.5 mm and about 5 mm, more preferably a width of about 150 μm and/or length of about 810 μm. (The length may be determined based on a balance between ensuring a robust pinning of the fluid (e.g., against vibrations or other forces that might disrupt the capillary pressure balance temporarily) and ensuring that hydraulic resistance of this section is not too high given that resistance is generally proportional to length). Generally, the retention valve 104 may have the greatest capillary pressure in the system; this may be enabled by having the smallest cross-sectional area for the fluid flow through the test strip. Advantageously, this may result in the valve not becoming empty of fluid during a test. Once fluid from the inlet flows into the channel, this valve may effectively pin the position of the fluid ‘plug’ at the inlet channel and may thereby prevent or reduce the introduction of air bubbles in the channel. From the retention valve 104, the fluid may flow into a microfluidic channel. This channel may have a width between about 10 μm and about 2000 μm. As discussed above, the cross sectional area of the channel will generally be greater than the cross sectional area of retention valve 104. For example, if the retention valve has a width of approximately 150 μm the microfluidic channel may have a width of approximately 250 μm.

The volume of reaction chamber 106 may be determined based on the assay reagents volumes. For example, in one embodiment reaction chamber 106 may have a width of approximately 1 mm while the surface area of the microfluidic cartridge (excluding the hydrophilic tape) in reaction chamber 106 may be approximately 11.5 mm². These dimensions may provide an approximate volume of 2.2 μL within chamber 106. In some embodiments, the dimensions of the components of the test strip may be determined such that the biosensing test is only performed on a portion of the substrate solution that undergoes oxidation in the presence of the bound enzyme, rather than all the substrate. Chamber 106 may be pre-functionalised with bioreceptor molecules such as antibodies and/or aptamers. The bioreceptor molecules may allow the analyte and conjugate binding reactions to occur in reaction chamber 106. In some embodiments, the bioreceptors are attached to the microfluidic cartridge cavity.

The channel following reaction chamber 106 may include a branched channel to test chamber 116. This channel may be initially closed if the test chamber vent 118 is sealed, and may thus reduce or prevent air being forced out by fluid. A small amount of fluid may still flow into this branch channel but will generally cease once the air pressure build-up compensates for any capillary pressure difference in the channel. The inlet of the test chamber 116 may comprise (e.g., be) a retention valve, which may be referred to as a test retention valve and may be a second retention valve 302 for example as shown in FIG. 3a. The test retention valve 302 may have a nominally equal cross-sectional area to that of the inlet microfluidic retention valve 104. Retention valve 302 may be filled from the main channel. In some embodiments, retention valve 302 preferably prevents fluids from emptying from this valve 302 into the measurement channel by forming a fluid ‘plug’ in a similar manner to inlet retention valve 104. In the absence of retention valve 302 some volume of unwanted solutions may enter the test chamber and may affect the later electrochemical measurement. Additionally or alternatively, retention valve 302 may reduce the likelihood of the formation of air bubbles in the channel.

Following retention valve 302, the channel can lead fluid toward an optional passive stop valve 114. Stop valve 114 may be shaped, for example, as an arrow or other shape where the channel width is increased. Stop valve 114 may be formed by a change in the hydrophilicity of the channel. Valve 114 may be placed close to the branching channel and/or distant from sensing chamber 116 to mitigate any contamination of sensing chamber 116 for example during the assay steps preceding the opening of the sensing chamber vent 118. Regardless of whether stop valve 114 is included, the branched measurement channel may include an optional constriction 304 prior to test chamber 116. Test chamber 116 may expose one or more test electrodes to a substrate solution in order to perform an electrochemical part of a biosensing test. Chamber 116 may be formed by a cut in the laminate layer.

Following the test chamber 116, there may be a vent channel 120 leading to a hydrophobic vent 118. The vent channel 120 may be included to reduce the effect of the hydrophobic vent hole 118 on the flow into the test chamber. (This may allow for simpler manufacture). However, in some embodiments vent hole 118 may be operatively connected directly or otherwise to chamber 116 (e.g., without vent channel 120). The vent hole 118, which may be a through-cut in the PMMA layer that is initially sealed by the vinyl label film, may be rendered hydrophobic by addition of a chlorinated organopolysiloxane thin film, for example, to the PMMA surface in this region. The vent 118 may be made hydrophobic to effectively stop, or significantly reduce rate of, flow of the solution. As a result, at the point of electrochemical measurement in the test chamber, a known volume of solution may have passed into the test chamber and the solution may be effectively or approximately stationary at the point of measurement. If the volume of passed solution is not controlled then the portion of the incubated solution from the reaction chamber may not be known and may cause errors and/or variability in the measurement, leading to less accurate and/or reliable results. If the solution is not stationary at the point of measurement then the accuracy of any measurement of the oxidised species in the substrate solution may be reduced as the solution may effectively be being replaced or refreshed during the measurement and the measurement regime no longer diffusion limited.

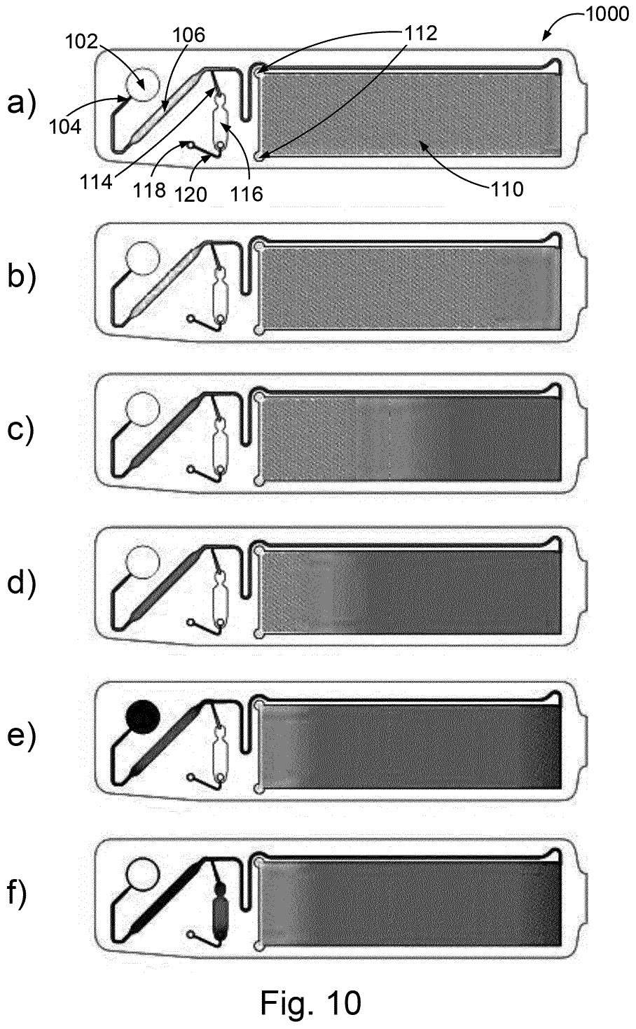

While the reaction(s), washing steps and/or substrate incubation in the reaction chamber take place (as discussed in more detail with reference to FIGS. 9 and 10), the test chamber vent 118 may be sealed to prevent or reduce flow into the test chamber 116. After the substrate incubation step in the reaction chamber 106, the user may be prompted, e.g. by software-controlled output from a saliva test system comprising the strip, to open the vent 118. This may be done by, for example, piercing the vinyl film with a pin or by peeling the vinyl film away from the cartridge. Other methods of breaking the seal and opening vent 118 may also be used.

A volume of solution may be retained in the inlet 102 of the test strip 300 to aid flow into the test chamber 116 on opening the test chamber vent 118. (In embodiments, this may allow a sufficiently large volume of substrate to be added to exceed the capacity of the test strip including the test chamber). Flow into the test chamber may not be capillary driven, for example when the hydrophilic tape is not present and/or the wide aperture in the laminate layer that forms the test chamber 116 has a reduced capillary pressure compared with the microfluidic channels. Additional hydrostatic pressure at the inlet 102 (from, for example, fluid in inlet 102) may then aid the flow of the solution from the reaction chamber 106 into the test chamber 116 once the vent 118 is opened.

As shown in FIG. 3b, capillary pump 110 may comprise an array of micropillars 308. In this embodiment, the micropillars 308 have a diamond or rhombic cross section that determines fluid flow path directions through the capillary pump, however other micropillar shapes may be utilised. Optionally, one or all of the corners of the cross sectional shape may be curved such that the cross section of micropillars 308 may only be approximately a diamond or rhombic cross section. The micropillars 308 may have a separation of between 5 μm and 750 μm. The distance between the pillars may be longer than the retention valve 104 width and/or shorter than the widths of any microchannels connecting inlet 102, reaction chamber 106 and capillary pump 110. For example, if retention valves 104, 302 have a width of about 150 μm and the microchannels have a width of about 250 μm, the pillars may have a separation of about 200 μm. It is notable that micropillar based capillary pumps may suffer from bypassing flows along a frame or boundary surrounding the array. In order to reduce or eliminate these bypassing flows, the capillary pump may have a peripheral clearance 312 of appropriate width. Preferably the width is greater than the smallest intra-micropillar distance, e.g., may be about 300 μm. However, the provision of a bypass channel may mean that a gap is formed between the micropillar array and pump inlet, and this may be a source of delay resulting in variation of a filling ‘front’ of the solutions moving across the capillary pump and/or potential air vesicle formation. To guide the liquid at the entrance of the capillary pump 110 towards the micropillar array 308 in a controllable fashion, while keeping the surrounding clearance frame 312 unfilled, a capillary pump inlet constriction 310 may be added. Such a constriction 310 may increase the tolerance of air vesicles that enter the pump 110, preferably without affecting filling flow rate and/or blocking the device. The capillary pump may also include two vent holes 112, wherein the second vent hole may further reduce the likelihood of blockages and/or aid in ensuring the capillary pump fills more uniformly. It is further notable that the super-hydrophilic properties of the hydrophilic tape combined with reduced surface tension of washing buffer(s) involved in the assays may make operation under low filling flow rates challenging.

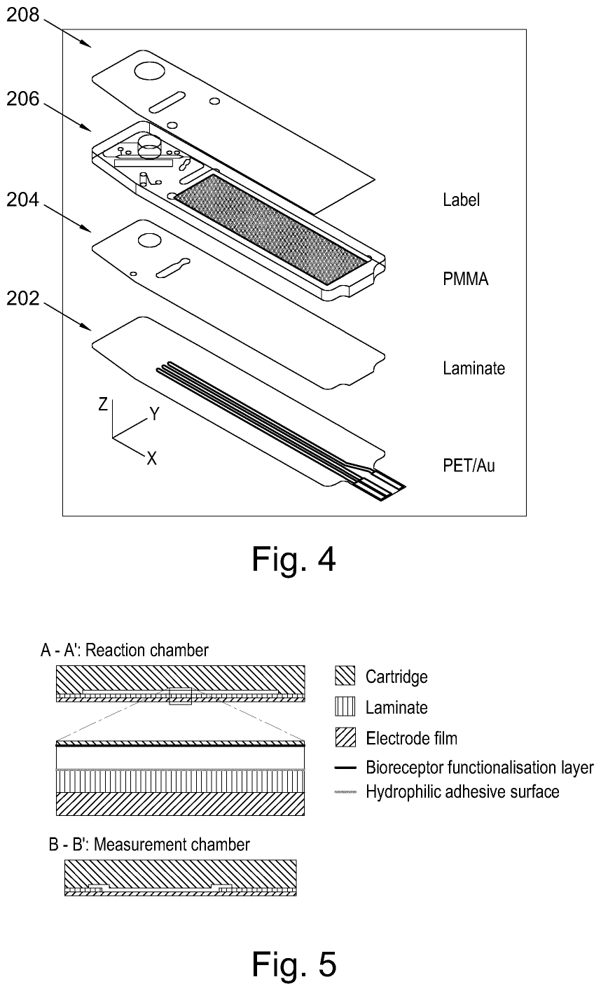

FIG. 4 shows an exploded view of the example test strip with four layers. The layers shown in this figure correspond to those of test strip 200. As shown in FIG. 4, layer 202 may form the base of the test strip, followed by layers 204, 206 and/or 208. The skilled person will understand that additional layers may also be provided, for example a base layer may be provided beneath layer 202 in order to protect the PET/Au film.

FIG. 5 shows a cross-sectional view of reaction chamber 106 along line A-A′ of FIG. 1 and test chamber 116 along line B-B′ of FIG. 1. Reaction chamber 106 may be formed between the second and third layers, for example between the laminate film and the PMMA surface. The chamber 106 may be formed by a channel engraved in the PMMA cartridge. The base of chamber 106 may be a hydrophilic adhesive surface of the laminate layer, and/or the ceiling of the chamber 106 may be the microfluidic cartridge layer. Bioreceptor molecules may be disposed on the portion of the PMMA cartridge forming the roof of chamber 106 in order to functionalise chamber 106.

Test chamber 116 may be formed between the first and third layers, for example between the PET/Au surface and the preferably unengraved PMMA surface. The chamber 116 may be formed by a through-cut, such as a slot, in the hydrophilic/hydrophobic laminate. The through-cut in the hydrophilic/hydrophobic laminate may overlap with the microfluidic network engraved in the PMMA layer at the inlet and/or the outlet of chamber 116. The design of the microfluidic network may reduce the hydrophobic barrier formed by the edge of the laminate that comes into contact with the liquid, and/or offer sufficient tolerance of misalignment between the PMMA layer and the laminate film.

The stop valve may be formed in the PMMA layer, and/or may comprise a widening in order to reduce capillary pressure (as shown in FIG. 3a). Such widening may occur over a relatively short distance and may thus cause a sudden drop in capillary pressure. The reduction in capillary pressure over the stop valve may reduce or prevent unwanted flow of solutions into the test chamber prior to opening the test chamber vent hole. Additionally or alternatively, resistance of the stop valve to fluid flow may be increased or caused by a change in the hydrophilicity or removal of the laminate film. The change in the hydrophilicity may be due to, for example, a change or removal of part of the hydrophilic adhesive film of the laminate layer. This can be seen in FIG. 5. However, resistance to fluid flow created by the stop valve may not be effective against the relatively large hydrostatic pressures that may occur when fluid is present in the inlet.

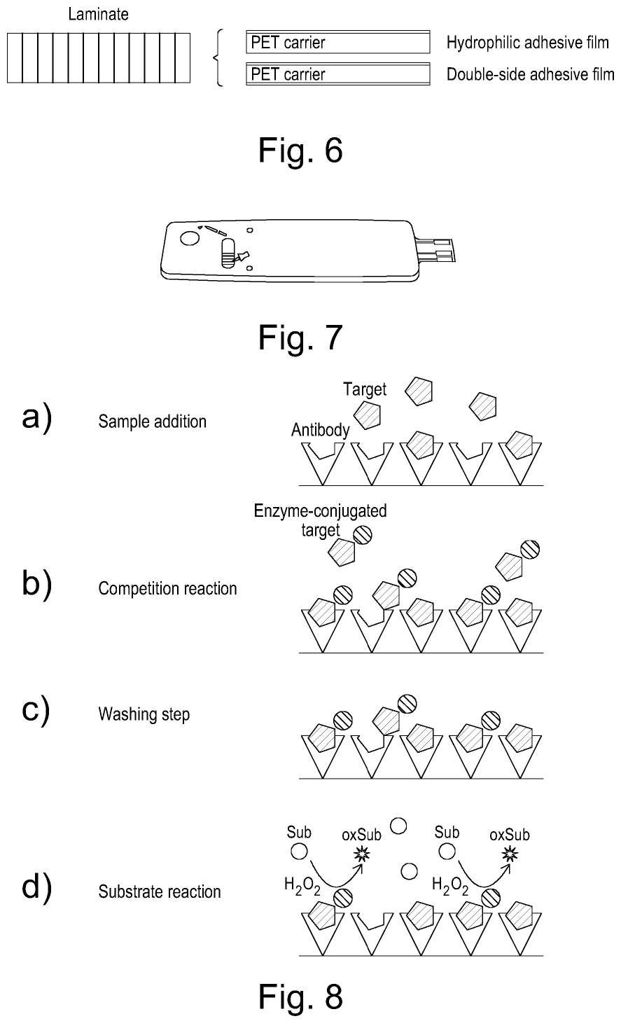

FIG. 6 shows an enhanced view of an example laminate layer 204 that may include a hydrophilic tape. Such tape may preferably have a water contact angle of below about 10 degrees and/or may help to increase the flow rate in channels in contact with the tape. Alternatively or additionally, adhesive tapes with greater water contact angles (for example, up to about 40 degrees) may be used, however this may reduce flow rate(s). However, as a consequence of this, a stationary fluid (which may be stationary because of, for example, exhausting fluid at the inlet and being retained by the retention valve) may bleed along the hydrophilic tape surface only. This may mean that the liquid-air meniscus does not advance but the fluid may wet the surface of the hydrophilic tape. This bleeding may reduce the fluid volume within the stationary fluid section until the point that the hydrophilic tape surface is wetted with fluid.

After the addition of the first solution (which may have a volume of, for example, 10 μL), the fluid flows into the channel and may be required to be resident in the reaction chamber for the duration of the first reaction (which may take, for example, up to 15 minutes). In this time, the fluid may bleed to coat the surface of the hydrophilic tape in the capillary pump area. This may result in the liquid-air meniscus receding. Due to the retention valve at the inlet, the liquid-air meniscus may move in the direction from the capillary pump towards the reaction chamber. To prevent the reaction chamber from drying out or partially drying out during the first incubation step, the initial volume added may be increased.

If later solutions are introduced in the device inlet, the liquid meniscus may start advancing again though the microfluidic network seamlessly. Any air bubbles that formed during the incubation experience may be subject to drag forces of sufficient magnitude to move them towards the capillary pump, where they may be absorbed without affecting the subsequent device operation.

FIG. 7 shows an example assembled test strip, wherein the test chamber vent may be connected directly to the test chamber.

FIG. 10 outlines an example operational flow of the above-mentioned process 1000 through example test strip 1000. Many features in test strip 1000 correspond to those shown in test strip 100 of FIG. 1, and the same reference numerals have been used for these features. We describe below each of steps a)-f), any one or more of which may be optional.

In step a), a sample (shown in blue) such as saliva is added into inlet 102. The sample flows through the test strip and into capillary pump 110 via retention valve 104, reaction chamber 106 and the connecting microfluidic channels. As capillary pump 110 fills with fluids, vent holes 112 may allow air in capillary pump to be displaced or escape. While in reaction chamber 106, target analytes (such as hormones) in the sample may bind to any bioreceptor molecules (such as antibodies) that were disposed in reaction chamber 106 during the test strip manufacturing process. The target analytes may bind to the bioreceptors through biorecognition. As the sample flows into the test strip, inlet 102 preferably becomes empty, however inlet retention valve 104 preferably retains a portion of the sample. Another portion of the sample may flow into the branching circuit comprising test chamber 116. However, this flow may be halted by a stop valve 114 constriction 304 (if either is present) and/or an increase in the pressure of the air trapped in the branching circuit.

In step b), a conjugate solution (shown in green) may be inserted into inlet 102. The conjugate solution may be, for example, a solution formed of the target analyte conjugated with an enzyme. The solution may flow into the test strip in the same way as the sample, displacing the sample through the test strip and into capillary pump 110. As with the sample, inlet 102 preferably becomes empty as the conjugate solution flows into the test strip, however retention valve 104 may again retain a portion of the conjugate solution. As the conjugate has displaced the sample, the sample may no longer be retained by retention valve 104. The conjugate may bind with at least some of the remaining unbound bioreceptor molecules. At this stage many (preferably most or all) of the bioreceptor molecules may be bound to either the target analyte or the conjugate.

In step c), a wash buffer solution (shown in red) may be placed in inlet 102. The wash buffer solution may displace the conjugate solution through the test strip and into the capillary pump 110. Advantageously, the wash buffer solution may reduce the number of unbound conjugate molecules in reaction chamber 106. This may prevent the removed conjugates from potentially reacting with later solutions and reducing the reliability of test results. Once again, inlet 102 preferably becomes empty as the wash buffer flows into the test strip, however retention valve 104 may retain a portion of the wash buffer solution. As the wash buffer has displaced the conjugate solution, the conjugate may then no longer be retained by retention valve 104.

In step d), one or more additional wash buffer solutions (also shown in red) are added to inlet 102. These additional buffers may displace the previous solutions as discussed in the previous steps. Each additional wash buffer inserted into the test strip may further reduce the number of unbound conjugate molecules in reaction chamber 106, further improving the accuracy and/or reliability of the test.

In step e), a substrate solution (shown in dark purple) is introduced into inlet 102. The substrate solution may flow through the test strip, displacing the previous solutions such as the wash buffer into capillary pump 110. Substrate solution in reaction chamber 106 may incubate by reacting with the bound enzyme-conjugate molecules. Such reaction may comprise oxidisation of the substrate solution. The incubated substrate solution is shown in FIG. 10 in light purple. The total volume of the fluids introduced to the test strip by this stage may exceed the total volume of the primary microfluidic circuit (i.e. the branched flow path excluding the branch comprising the test chamber). This may ensure that there is remaining solution in inlet 102 during the substrate incubation. When the inlet 102 is thus not empty, this may increase the hydrostatic pressure in the test strip and may thereby aid the flow of the substrate solution into test chamber 116.

After a preferably predetermined incubation period, the user may open hydrophobic test chamber valve 118 to allow the substrate solution to flow into test chamber 116, as shown in step f). This flow may result in inlet 102 becoming fully or partially empty, however a portion of the substrate solution is preferably retained by inlet retention valve 104. The substrate solution may continue to flow through vent channel 120 towards hydrophobic vent hole 118. Advantageously, the hydrophobic nature of vent hole 118 may slow or temporarily halt the flow of the substrate solution in test chamber 116. A stationary or slow flowing solution will generally produce more accurate results than a faster flowing solution. In some embodiments, the volume of substrate solution and/or positioning or arrangement of the branching microfluidic system may be such that all of the reacted substrate solution enters test chamber 116, as shown in step f) of FIG. 10. The test electrodes may then be controlled (for example, by means of a reader device or other computing device such as a general purpose computer or mobile computing device) to perform an electrochemical transduction. Generally, for a given incubation period, the greater the proportion of bound enzyme-conjugates the more reacted substrate solution will be produced. As the analytes in the conjugate solution and the sample compete for binding spots, in general, an increase in the amount of reacted substrate produced in a given period of time may correlate with a decrease in the levels of the target analyte in the saliva. Therefore, by testing the incubated solution using the test strip of an embodiment, it may be possible to determine a level of the target analyte in the sample. FIG. 12 shows a block diagram of an example sample collection device 1200. The sample collection device may comprise an inlet 1202 for receiving a sample, a storage chamber 1206 for storing a received sample and an outlet 1206 for inserting the sample into a test strip. The collector device may additionally or alternatively be used to insert other fluids, such as a substrate solution or wash buffer, into the test strip. In some embodiments, the inlet 1202 may also be the outlet 1206. Storage chamber 1206 may be designed to hold a suitable volume of a sample for use in a test strip.

FIG. 13 shows a block diagram of an example test system 1300 comprising a test strip 1302, a collection device 1304 and a reader device 1306. The test strip 1302 may be, for example, test strip 100 of FIG. 1. Similarly, collection device 1304 and reader device 1306 may be collection device 1200 of FIG. 12 and reader device 1100 of FIG. 11 respectively.

1. An integrated fluid sample test strip comprising:
i. an inlet for receiving a series of solutions, said solutions comprising at least a fluid sample and a substrate solution, wherein the inlet comprises a retention valve for temporarily retaining each said solution to thereby reduce air flow through the retention valve;
ii. a reaction chamber to receive the solutions from the inlet via the retention valve, the reaction chamber functionalized with one or more bioreceptors for binding to a target analyte;
iii. a capillary pump to receive from the reaction chamber at least one of the solutions including at least the fluid sample, the capillary pump comprising at least one vent hole to allow any air to escape from the capillary pump and thereby reduce pressure in the capillary pump;
iv. a test chamber to receive the substrate solution from the reaction chamber, the test chamber comprising a plurality of test electrodes to perform at least part of a biosensing test of the substrate solution; and
v. a hydrophobic vent hole coupled to the test chamber to allow a flow of solution from the reaction chamber into the test chamber when the vent hole is unsealed and to allow a flow of solution from the reaction chamber to the capillary pump when the vent hole is sealed.

2. The test strip of claim 1, comprising a branched flow path to guide solution from the inlet to the hydrophobic vent hole and from the inlet to the capillary pump, wherein the capillary pump comprises at least one capillary channel, the branched flow path comprising at least the elements i-v including the at least one capillary channel,
wherein a smallest cross-sectional area of the branched flow path is a cross-sectional area of the retention valve.

3. The test strip of claim 1, wherein the reaction chamber is configured to incubate a solution and the test strip comprises a further retention valve for temporarily retaining a said incubated solution.

4. The test strip of claim 1, wherein the capillary pump comprises at least one capillary channel defined by an array of micropillars.

5. The test strip of claim 4, wherein at least one said micropillar comprises a substantially diamond-shaped cross section.

6. The test strip of claim 4, wherein the capillary pump comprises a bypass channel along at least part of a perimeter of the capillary pump, wherein a smallest cross-sectional width of the bypass channel is greater than a smallest separation of between adjacent said micropillars.

7. The test strip of claim 4, wherein a smallest separation between adjacent said micropillars is less than a smallest width of a solution flow path from the reaction chamber to the capillary pump.

8. The test strip of claim 1 wherein the capillary pump has an inlet comprising a constriction.

9. The test strip of claim 1 and comprising a vent hole channel, wherein the hydrophobic vent hole is coupled to the test chamber via the vent hole channel to allow any air in the test chamber to escape to thereby reduce pressure in the test chamber.

10. The test strip of claim 1, comprising at least one of:
a hydrophilic layer, wherein at least one surface of the hydrophilic layer is hydrophilic; and
a polymer layer.

11. The test strip of claim 10, wherein:
the test chamber is formed in at least the hydrophilic layer;
a channel for guiding solution from the reaction chamber to the capillary pump is formed in at least the polymer layer;
the inlet is formed at in least the polymer layer;
the capillary pump is formed in at least the polymer layer; and/or
at least one said vent hole is formed in at least the polymer layer.

12. The test strip of claim 1, comprising a passive stop valve to at least reduce a flow rate of solution into the test chamber.

13. The test strip of claim 1, wherein the fluid sample comprises saliva, blood, blood serum, blood plasma, urine, nasal fluid or solutions thereof.

14. The test strip of claim 1, configured to measure levels of the analyte, wherein the analyte is a hormone.

15. The test strip of claim 1, configured to perform an ELISA or ELONA test.

16. A fluid sample test system comprising the fluid sample test strip of claim 1 and at least one of:
a fluid sample collector device for collecting the fluid sample and inputting the fluid sample into the inlet; and
a reader device for controlling at least one of the test electrodes to perform the at least part of the biosensing test, and to output a result of the biosensing test.

17. Use of the test strip of claim 1 or the test system of claim 16, to perform an ELISA or ELONA test.

18. The use according to claim 17, comprising:
(i) receiving the fluid sample in the inlet;
(ii) receiving the substrate solution in the inlet; and/or
(iii) unsealing the vent hole.

19. The use according to claim 18, comprising:
(iv) receiving a solution comprising an enzyme-conjugate in the inlet; and/or
(v) receiving one or more wash-buffer solutions in the inlet.
